(19) United States
(12) Patent Application Publication
(10) Pub. No.: US 2026/0368888 A1
(43) Pub. Date: Nov. 19, 2026

(54) METHOD AND APPARATUS FOR PROVIDING PROSTATE CANCER RISK ASSESSMENT INFORMATION USING PROBABILISTIC SEGMENTATION BASED SOFT PROSTATE VOLUME AND UNCERTAINTY REFLECTED RISK WEIGHTED PSA DENSITY

(71) Applicant: UIF (UNIVERSITY INDUSTRY FOUNDATION), YONSEI UNIVERSITY, Seoul (KR)

(72) Inventors: Jongsoo LEE, Seoul (KR); Jongjin YOON, Seoul (KR); Hyunho HAN, Seoul (KR); John Jang-Hwan KIM, Seoul (KR); Seung Hwan LEE, Seoul (KR); Kyung Tak OH, Seongnam-si (KR)

(73) Assignee: UIF (UNIVERSITY INDUSTRY FOUNDATION), YONSEI UNIVERSITY, Seoul (KR)

(21) Appl. No.: 19/576,623

(22) Filed: Mar. 24, 2026

(30) Foreign Application Priority Data
Apr. 2, 2025 (KR) 10-2025-0042767
Jan. 15, 2026 (KR) 10-2026-0008279

(51) Int. Cl. G06T 7/00; G06T 7/11; G16H 50/30

(52) U.S. Cl. CPC G06T 7/0012; G06T 7/11; G16H 50/30; G06T 2207/10028; G06T 2207/10081; G06T 2207/10088; G06T 2207/20112; G06T 2207/20172; G06T 2207/30081

(57) ABSTRACT

The present disclosure relates to a method and an apparatus for providing prostate cancer risk assessment information based on medical imaging data and a serum prostate-specific antigen (PSA) level of a patient. More particularly, the present disclosure relates to a method and an apparatus for generating and outputting risk-weighted prostate-specific antigen density (Risk-Weighted PSAD) information reflecting a confidence interval, by calculating a soft prostate volume and a voxel-wise uncertainty map of a prostate using a probabilistic segmentation model.

### CROSS-REFERENCE TO RELATED APPLICATION

This application claims priority to and the benefit of Korean Patent Application No. 10-2025-0042767, filed on Apr. 2, 2025, and Korean Patent Application No. 10-2026-0008279, filed on Jan. 15, 2026, the disclosure of which is incorporated herein by reference in its entirety.

### BACKGROUND

### 1. Field of the Invention

The present disclosure relates to a method and an apparatus for providing prostate cancer risk assessment information based on medical imaging data and a serum prostate-specific antigen (PSA) level of a patient. More particularly, the present disclosure relates to a method and an apparatus for generating and outputting risk-weighted prostate-specific antigen density (Risk-Weighted PSAD) information reflecting a confidence interval, by calculating a soft prostate volume and a voxel-wise uncertainty map of a prostate using a probabilistic segmentation model.

### 2. Discussion of Related Art

Early diagnosis and risk assessment of prostate cancer are clinically very important tasks, and a serum prostate-specific antigen (PSA) level is a representative indicator widely used in prostate cancer screening tests. However, the PSA level may also increase due to various non-malignant factors such as prostatitis and benign prostatic hyperplasia. Accordingly, relying solely on the PSA level has limitations in accurately determining a risk of prostate cancer.

To address these limitations, prostate-specific antigen density (PSA density, PSAD), which adjusts the PSA level by prostate volume, has been used in clinical practice. Because PSAD reflects that, even for the same PSA level, the risk may be relatively higher when the prostate volume is small, PSAD may provide improved diagnostic usefulness compared with the PSA level alone. However, the reliability of PSAD largely depends on the accuracy and consistency of prostate volume estimation.

Prostate volume is often estimated from medical imaging such as magnetic resonance imaging (MRI) or computed tomography (CT). In clinical practice, approaches such as manual contour delineation by a reader or simplified geometric approximation models are commonly used. However, manual contour delineation may lead to inter-reader variability and reduced reproducibility depending on examination conditions or reader expertise. In addition, calculations based on approximation models may fail to sufficiently reflect irregular shapes of the prostate.

In this regard, attempts have been increasing to automatically segment the prostate region using deep learning-based segmentation models and to estimate the prostate volume based on the segmentation results. However, many automated segmentation approaches calculate the volume by binarizing voxels as belonging either to the prostate region or to a non-prostate region. In such cases, volume estimation errors may increase due to a partial volume effect occurring in voxels located at the boundary of the prostate region. In particular, uncertainty in boundary voxels may increase depending on image resolution, slice thickness, and imaging angle, thereby reducing stability of volume estimation based on binarization.

In addition, prostate segmentation results may be affected by prediction uncertainty of the model, and predicted probabilities may vary during the inference process of the model even for the same image. Nevertheless, many existing approaches do not systematically incorporate uncertainty of segmentation results into the volume estimation process or fail to quantitatively present how such uncertainty affects the final PSAD value. As a result, there may be insufficient basis for determining how reliable the finally provided PSAD value is.

Clinically, PSAD results around a specific threshold may have a significant influence on decision-making. If errors in volume estimation cause PSAD to be overestimated or underestimated, unnecessary additional examinations may be performed or necessary examinations may be delayed. Accordingly, it is important to consider estimation errors and uncertainty in prostate volume during calculation of PSAD and to present the results in a form interpretable by decision makers. However, conventional techniques often fail to systematically support quantitative uncertainty-based correction or provision of a confidence interval.

Furthermore, medical imaging data may vary in quality and characteristics depending on imaging devices, imaging parameters, patient posture, and hospital-specific protocols. Such variability may increase ambiguity of prostate boundaries and degrade performance of model-based segmentation. In particular, in regions where boundaries are unclear, binarization-based volume estimation tends to amplify small segmentation deviations into large volume deviations, thereby further aggravating reliability issues of PSAD.

In addition, in order to improve reliability of prostate cancer risk assessment, it is desirable to provide not only a single PSAD value but also a conservative interpretation reflecting a degree of uncertainty together with the basis on which the PSAD value is derived. However, conventional techniques do not sufficiently present a framework that integrates prostate volume estimation and PSAD calculation within a consistent uncertainty framework, such that soft volume estimation, uncertainty accumulation, confidence interval derivation, and generation of risk-weighted PSAD are provided in a linked manner.

Accordingly, there is a need for a method and an apparatus capable of deriving a soft prostate volume and a corresponding confidence interval by reflecting probabilistic segmentation results and prediction uncertainty of prostate medical imaging data in a volume estimation process, and providing risk-weighted PSA density information useful for assessing a risk of prostate cancer based on the derived results.

### SUMMARY OF THE INVENTION

The present disclosure is intended to solve the above-mentioned problems and has the following objectives.

Various embodiments of the present disclosure provide a method and an apparatus for improving reliability of prostate volume estimation and prostate-specific antigen density (PSA density) by quantitatively reflecting uncertainty of prostate segmentation results based on medical imaging. Various embodiments of the present disclosure reduce volume estimation errors caused by partial volume effects of boundary voxels in binarization-based volume estimation, and provide risk-weighted PSA density information meaningful for clinical decision-making.

Various embodiments of the present disclosure generate three-dimensional voxel data of a prostate based on two-dimensional medical imaging data acquired from magnetic resonance imaging (MRI) or computed tomography (CT), and derive a membership probability map of a prostate region and a voxel-wise uncertainty map using a probabilistic segmentation model. Instead of applying threshold-based binarization to boundary voxels of the prostate region, various embodiments of the present disclosure calculate a soft prostate volume using a continuous weighting function that jointly reflects membership probability and uncertainty.

Various embodiments of the present disclosure derive a confidence interval of prostate volume based on a total amount of uncertainty accumulated during calculation of the soft prostate volume, thereby providing not only a single volume value but also information reflecting reliability of the result. Various embodiments of the present disclosure further provide a method and an apparatus for generating and outputting risk-weighted prostate-specific antigen density (Risk-Weighted PSAD) information by combining a serum PSA level with the soft prostate volume reflecting the confidence interval, thereby providing a more robust criterion for assessing a risk of prostate cancer.

Various embodiments of the present disclosure improve interpretability of the uncertainty map through approaches such as probabilistic inference-based uncertainty estimation, utilization of uncertainty metrics based on predictive entropy or mutual information, and representation of separated uncertainty components. Various embodiments of the present disclosure further provide a method and an apparatus capable of identifying boundary regions having high uncertainty, or enabling conservative risk assessment by incorporating uncertainty-based adjustment terms into the confidence interval and the risk-weighted PSA density.

Various embodiments of the present disclosure improve clinical workflow efficiency by automating estimation of prostate volume and PSA density, thereby reducing inter-reader variability and providing consistent results even under varying imaging environments. In addition, various embodiments of the present disclosure provide not only numerical results but also probability and uncertainty information serving as the basis for the results, thereby enabling medical professionals to interpret prostate cancer risk more transparently and reliably and to perform clinical decision-making more effectively.

The technical problems to be solved by The present disclosure are not limited to those mentioned above, and other technical problems not mentioned herein will be clearly understood by those skilled in the art from the following description.

According to various embodiments of The present disclosure, there is provided a method performed by an electronic device. The electronic device comprises an input device, an output device, a processor, and a memory. The method comprises: acquiring, by the input device, two-dimensional medical image data related to a prostate of a patient from a magnetic resonance imaging (MRI) device or a computed tomography (CT) device; generating, by the processor, three-dimensional voxel data of the prostate based on the two-dimensional medical image data; applying, by the processor, a probabilistic segmentation model to the three-dimensional voxel data to generate (i) a membership probability map representing, for each voxel, a probability that the voxel belongs to a prostate region as a continuous value between 0 and 1, and (ii) a voxel-wise uncertainty map representing a degree of prediction uncertainty of the membership probability map; calculating, by the processor, a soft prostate volume by integrating continuous values of the membership probability map using a continuous weighting function defined such that (i) the weighting function is proportional to values of the membership probability map and (ii) the weighting function is inversely proportional to values of the voxel-wise uncertainty map, for voxels located at a boundary of the prostate region without performing threshold-based binarization; determining, by the processor, a confidence interval of the calculated soft prostate volume based on a total amount of uncertainty accumulated during calculation of the soft prostate volume; generating, by the processor, risk-weighted prostate specific antigen density (Risk-Weighted PSAD) information serving as a criterion for assessing a prostate cancer risk, based on a serum prostate specific antigen level of the patient and the soft prostate volume reflecting the confidence interval; and outputting, by the output device, the risk-weighted prostate specific antigen density information.

According to various embodiments of The present disclosure, there is provided an electronic device. The electronic device comprises: an input device; an output device; a processor; and a memory storing instructions executable by the processor. The instructions cause the processor to perform operations comprise: acquiring, by the input device, two-dimensional medical image data related to a prostate of a patient from a magnetic resonance imaging (MRI) device or a computed tomography (CT) device; generating, by the processor, three-dimensional voxel data of the prostate based on the two-dimensional medical image data; applying, by the processor, a probabilistic segmentation model to the three-dimensional voxel data to generate (i) a membership probability map representing, for each voxel, a probability that the voxel belongs to a prostate region as a continuous value between 0 and 1, and (ii) a voxel-wise uncertainty map representing a degree of prediction uncertainty of the membership probability map; calculating, by the processor, a soft prostate volume by integrating continuous values of the membership probability map using a continuous weighting function defined such that (i) the weighting function is proportional to values of the membership probability map and (ii) the weighting function is inversely proportional to values of the voxel-wise uncertainty map, for voxels located at a boundary of the prostate region without performing threshold-based binarization; determining, by the processor, a confidence interval of the calculated soft prostate volume based on a total amount of uncertainty accumulated during calculation of the soft prostate volume; generating, by the processor, risk-weighted prostate specific antigen density (Risk-Weighted PSAD) information serving as a criterion for assessing a prostate cancer risk, based on a serum prostate specific antigen level of the patient and the soft prostate volume reflecting the confidence interval; and outputting, by the output device, the risk-weighted prostate specific antigen density information.

According to various embodiments of The present disclosure, there is provided a non-transitory computer-readable storage medium having stored thereon instructions that, when executed by at least one processor, cause the at least one processor to perform operations, wherein the operations comprise the steps of the method performed by the electronic device according to various embodiments of The present disclosure.

### BRIEF DESCRIPTION OF THE DRAWINGS

FIG. 1 illustrates an overview of an operation of an electronic device according to various embodiments of the present disclosure.

FIG. 2 illustrates a configuration of an electronic device according to various embodiments of the present disclosure.

FIG. 3 illustrates a method of operating an electronic device according to various embodiments of the present disclosure.

FIG. 3A illustrates a method of operating an electronic device according to various embodiments of the present disclosure.

FIG. 4 illustrates a structure of a multi-layer perceptron (MLP) for implementing a deep learning algorithm according to various embodiments of the present disclosure.

FIG. 5 illustrates an example of a preprocessing process of image data before the image data is input to an artificial intelligence model according to various embodiments of the present disclosure.

FIG. 6 illustrates an example of an artificial intelligence model for detecting a prostate region according to various embodiments of the present disclosure.

FIG. 7 illustrates an example of a 3D U-Net structure (MRI image input example) according to various embodiments of the present disclosure.

FIG. 8 illustrates an example of an output screen of an electronic device for providing prostate detail information based on an artificial intelligence model according to various embodiments of the present disclosure.

### DETAILED DESCRIPTION OF EXEMPLARY EMBODIMENTS

Hereinafter, embodiments of The present disclosure will be described in detail with reference to the accompanying drawings so that those skilled in the art may easily practice The present disclosure. It should be understood that The present disclosure may be embodied in different ways and is not limited to the following embodiments.

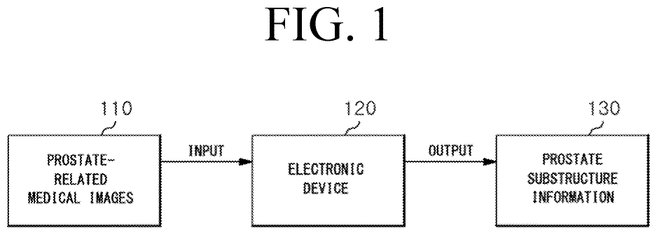

FIG. 1 illustrates an overview of an operation of an electronic device according to various embodiments of the present disclosure.

Referring to FIG. 1, an electronic device 120 according to various embodiments of the present disclosure operates in a structure in which prostate-related medical imaging data 110 is received as input and prostate detail information 130 is output. The prostate detail information 130 may include at least one of prostate volume information and prostate-specific antigen density (PSA density) information.

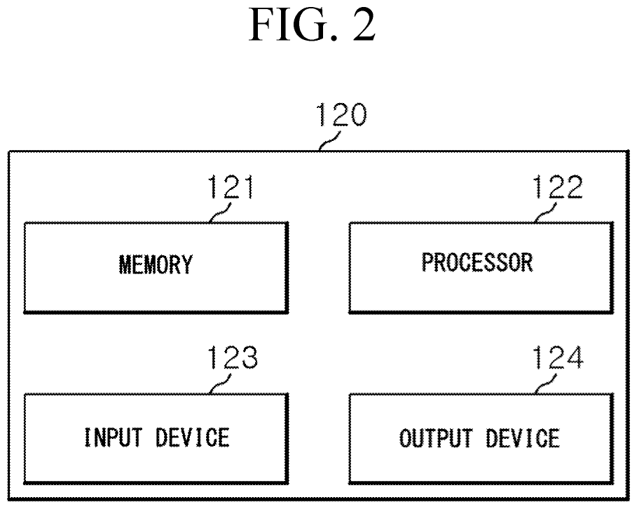

FIG. 2 illustrates a configuration of an electronic device according to various embodiments of the present disclosure.

Referring to FIG. 2, the electronic device 120 includes a memory 121, a processor 122, an input device 123, and an output device 124.

The memory 121 may be connected to the processor 122, the input device 123, and the output device 124, and may store information input through the input device 123. The memory 121 may also be connected to the processor 122 and may store data such as a basic program for operation of the processor 122, application programs, configuration information, and information generated through operations of the processor 122. The memory 121 may include a volatile memory, a non-volatile memory, or a combination of a volatile memory and a non-volatile memory. The memory 121 may provide stored data according to a request from the processor 122.

The processor 122 may be configured to implement procedures and or methods proposed in the present disclosure. The processor 122 may control overall operations of the electronic device 120. For example, the processor 122 may record data in the memory 121 and read data from the memory 121. The processor 122 may also receive information through the input device 123 and may output information through the output device 124. The processor 122 may include at least one processor.

The input device 123 may be connected to the processor 122 and may receive input information. The input device 123 may include, for example, a touch display, a keypad, a keyboard, or an information input module. According to an embodiment, the electronic device 120 may further include a transceiver, and the input device 123 may receive information from another device connected through a wired or wireless communication network via the transceiver.

The output device 124 may be connected to the processor 122 and may output information in a form of video or audio. The output device 124 may include, for example, a display, a speaker, or an information output module. According to an embodiment, the electronic device 120 may further include a transceiver, and the output device 124 may transmit and output information to another device connected through a wired or wireless communication network via the transceiver.

Although not illustrated in FIG. 2, according to an embodiment, the electronic device 120 may further include a transceiver. The transceiver may be connected to the processor 122 and may transmit and or receive signals. All or part of the transceiver may be referred to as a transmitter, a receiver, or a transceiver.

The transceiver may support at least one of various communication standards including wired access systems and wireless access systems such as an IEEE 802.xx system of the Institute of Electrical and Electronics Engineers, an IEEE Wi-Fi system, a 3GPP system, a 3GPP LTE system, a 3GPP 5G NR system, a 3GPP 6G system, a 3GPP2 system, or Bluetooth.

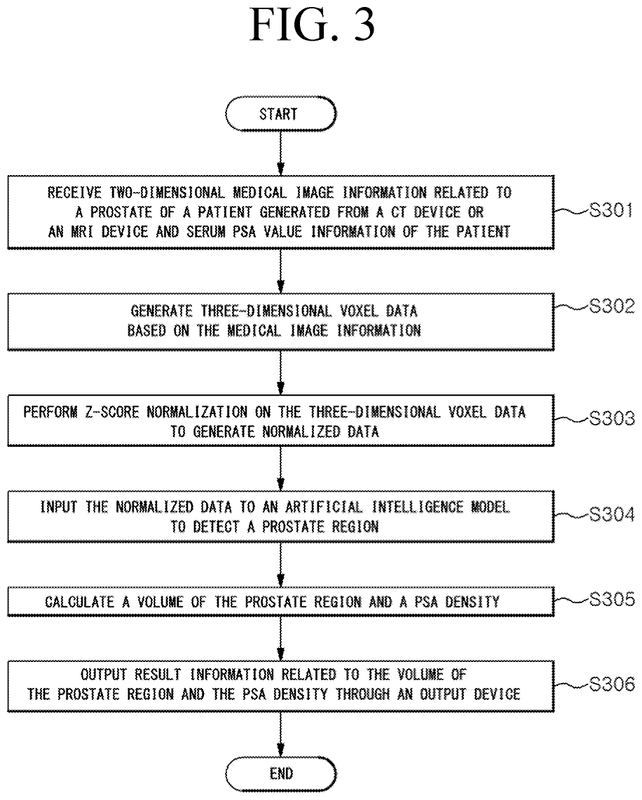

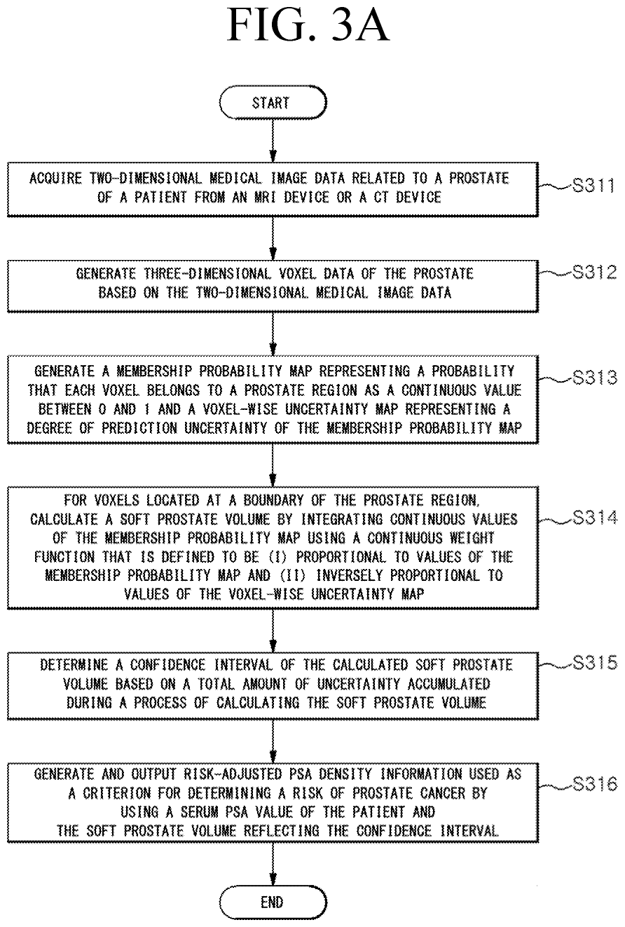

FIG. 3 illustrates a method of operating an electronic device according to various embodiments of the present disclosure.

Referring to FIG. 3, an embodiment of FIG. 3 illustrates an example of a method performed by an electronic device for providing prostate detail information including at least one of prostate volume information and prostate-specific antigen density (PSAD) information based on a prostate image. The electronic device may include an input device, an output device, a processor, and a memory.

In step S301, the electronic device receives, through the input device, two-dimensional medical imaging information related to a prostate of a patient generated by a computed tomography (CT) device or a magnetic resonance imaging (MRI) device, and serum prostate-specific antigen level (serum PSA level) information of the patient.

In step S302, the electronic device generates, by the processor, three-dimensional voxel data based on the medical imaging information.

In step S303, the electronic device generates normalized data by performing Z-score normalization on the three-dimensional voxel data by the processor.

In step S304, the electronic device detects a prostate region by the processor by inputting the normalized data to an artificial intelligence model stored in the memory.

In step S305, the electronic device calculates, by the processor, a prostate volume and the PSA density for the prostate region.

In step S306, the electronic device outputs, through the output device, result information related to the prostate volume and the PSA density for the prostate region.

According to various embodiments of the present disclosure, the three-dimensional voxel data may be generated by combining multiple two-dimensional image slices based on the medical imaging information.

According to various embodiments of the present disclosure, the prostate region may be detected by performing two-dimensional object detection on each of the multiple image slices based on the medical imaging information and integrating detected two-dimensional prostate regions from the respective slices into three-dimensional masking data.

According to various embodiments of the present disclosure, the artificial intelligence model may be based on a convolutional neural network (CNN). The artificial intelligence model may be trained to calculate the prostate volume for the prostate region.

According to various embodiments of the present disclosure, the PSA density may be calculated based on the prostate volume of the prostate region and the serum PSA level.

According to various embodiments of the present disclosure, step S302 of generating the three-dimensional voxel data based on the medical imaging information may include extracting, by the processor, resolution information of the multiple image slices and spacing information between the multiple image slices from metadata of the medical imaging information, determining, by the processor, a region of interest (ROI) based on a central prostate region of the multiple image slices, and generating the three-dimensional voxel data by correcting information within the ROI of the multiple image slices based on a multi-level resampling algorithm.

According to various embodiments of the present disclosure, the Z-score normalization may be configured to remove outliers and noise of the three-dimensional voxel data by filtering based on a comparison between a global average and a local average for each pixel value of the three-dimensional voxel data. The normalized data may be divided by the processor into patches corresponding to units of the ROI. The divided patches of the normalized data may be input to the artificial intelligence model with weighted priority assigned according to a detection probability of the prostate region for training of the artificial intelligence model.

According to various embodiments of the present disclosure, the normalized data may be dynamically divided into patch units by the processor based on the size of the data. In order to optimize a training speed of the artificial intelligence model, the normalized data may be divided into patch units and input to the artificial intelligence model in a parallel computation manner. Among the divided patches of the normalized data, patches having a high probability of including the prostate region may be preferentially input to the artificial intelligence model for training. Different priority weights may be assigned to respective patches in order to improve detection accuracy of the prostate region by the artificial intelligence model.

According to various embodiments of the present disclosure, among the divided patches, patches having a high probability of including the prostate region may be determined by scoring the likelihood of inclusion of the prostate region based on texture heterogeneity and contrast within a specific region of interest (ROI) of the prostate.

According to various embodiments of the present disclosure, patches having a high probability of including the prostate region may correspond to patches having a likelihood score higher than a threshold suspicion score.

In relation to the region of interest (ROI), the ROI may refer to a specific region in which the prostate region is likely to be included in a prostate image. The ROI may be determined based on patterns of a prostate structure learned by the artificial intelligence model or may be defined according to an assessment of a medical imaging expert.

With respect to patch division within the ROI, the entire region may be divided into multiple patches and each patch may be independently evaluated to score the likelihood of including the prostate region. A patch size and a division method may be determined according to analysis resolution and computational efficiency.

With respect to evaluation based on texture heterogeneity, texture heterogeneity refers to a degree of pattern irregularity or complexity among pixels within a patch. In general, the prostate region may exhibit texture characteristics different from surrounding tissues.

In relation to heterogeneity evaluation methods, a gray-level co-occurrence matrix (GLCM) may evaluate texture heterogeneity by analyzing a distribution and correlation of pixel intensity levels. Entropy may quantify heterogeneity by evaluating randomness or irregularity of pixels within a patch. Variance may detect high-contrast regions by calculating dispersion of pixel values within a patch.

With respect to contrast-based evaluation, contrast indicates a difference in intensity between the prostate region and surrounding tissues, which is an important factor for emphasizing characteristic patterns of the prostate in medical images.

In relation to contrast evaluation methods, contrast may be quantified by measuring a difference between maximum and minimum pixel values within a patch as global contrast. Local contrast evaluation may further distinguish boundaries between the prostate region and surrounding tissues.

With respect to scoring and patch determination, each patch may be assigned a score based on texture heterogeneity and contrast. For example, a final score may be calculated by a weighted average of a texture heterogeneity score and a contrast score. Patches having high scores may be determined as having a high probability of including the prostate region.

With respect to patch selection, among the scored patches, patches having a highest score or patches exceeding a threshold may be selected. These selected patches may be preferentially input to the artificial intelligence model to improve detection accuracy of the prostate region.

The method proposed in the present disclosure may provide the following effects.

In relation to computational efficiency, only patches having a high probability of including the prostate region may be selected and input to the artificial intelligence model instead of analyzing an entire image, thereby significantly reducing processing time and computational cost.

In relation to detection accuracy, scoring based on texture heterogeneity and contrast may more clearly distinguish characteristics of the prostate region and contribute to improving detection performance and accuracy of the artificial intelligence model.

In relation to assisting medical practitioners, detected patches may allow medical practitioners to quickly identify a location and shape of the prostate, thereby efficiently supporting a diagnostic process.

The patch determination method for identifying patches having a high probability of including the prostate region proposed in the present disclosure provides an innovative approach of evaluating and scoring patches based on texture heterogeneity and contrast. Through this approach, training and prediction accuracy of the artificial intelligence model may be maximized, and efficiency and reliability of prostate cancer diagnosis may be significantly improved.

According to various embodiments of the present disclosure, the filtering may be configured to apply a combination of a Gaussian filter and an average filter in order to remove high-frequency noise. The normalized data to which the filtering is applied may be input to the artificial intelligence model.

According to various embodiments of the present disclosure, the embodiment of FIG. 3 may further include receiving past information related to the prostate of the patient through the input device.

According to various embodiments of the present disclosure, the artificial intelligence model may be configured to analyze changes in the prostate volume and the PSA density based on the normalized data.

According to various embodiments of the present disclosure, the result information may be configured to visually provide a trend of change based on comparison with the past information with respect to changes in size and location of the prostate region.

According to various embodiments of the present disclosure, the result information may include information on the prostate volume of the prostate region, information on the PSA density, and two-dimensional or three-dimensional visualized graphic information of the prostate region.

According to various embodiments of The present disclosure, an electronic device for providing prostate substructure information may include an input device, an output device, a transceiver, a processor, and a memory, wherein the memory stores instructions for operations executed by the processor, and the operations may be configured to perform the method of operation of the electronic device according to the embodiment of FIG. 3.

According to various embodiments of The present disclosure, a non-transitory computer-readable storage media storing instructions that, when executed by at least one processor, cause the at least one processor to perform operations may be provided, wherein the operations include the method of operation of the electronic device according to the embodiment of FIG. 3.

FIG. 3A illustrates a method of operating an electronic device according to various embodiments of the present disclosure.

In the embodiment of FIG. 3A, the electronic device includes an input device, an output device, a processor, and a memory.

Referring to FIG. 3A, in step S311, the electronic device acquires, by the input device, two-dimensional medical image data related to a prostate of a patient from a magnetic resonance imaging (MRI) device or a computed tomography (CT) device.

In step S312, the electronic device generates, by the processor, three-dimensional voxel data of the prostate based on the two-dimensional medical image data.

In step S313, the electronic device applies, by the processor, a probabilistic segmentation model to the three-dimensional voxel data to generate a membership probability map representing, for each voxel, a probability that the voxel belongs to a prostate region as a continuous value between 0 and 1, and a voxel-wise uncertainty map representing a degree of prediction uncertainty of the membership probability map.

In step S314, the electronic device calculates, by the processor, a soft prostate volume by integrating continuous values of the membership probability map using a continuous weighting function defined such that (i) the continuous weighting function is proportional to values of the membership probability map and (ii) the continuous weighting function is inversely proportional to values of the voxel-wise uncertainty map, for voxels located at a boundary of the prostate region without performing threshold-based binarization.

In step S315, the electronic device determines, by the processor, a confidence interval of the calculated soft prostate volume based on a total amount of uncertainty accumulated during calculation of the soft prostate volume.

In step S316, the electronic device generates, by the processor, risk-weighted prostate specific antigen density (Risk-Weighted PSAD) information serving as a criterion for assessing a prostate cancer risk, based on a serum prostate specific antigen level of the patient and the soft prostate volume reflecting the confidence interval, and outputs, by the output device, the risk-weighted prostate specific antigen density information.

This aspect constructs three-dimensional voxel data of the prostate based on medical image data, and jointly generates a membership probability map and a voxel-wise uncertainty map by applying a probabilistic segmentation model. In this configuration, the membership probability map is a probabilistic map expressing, as a continuous value between 0 and 1, a probability that each voxel belongs to the prostate region, and the voxel-wise uncertainty map is a map expressing, on a voxel-by-voxel basis, how much the corresponding probability prediction fluctuates.

This aspect has an effect of naturally reflecting partial contributions of boundary voxels by excluding threshold-based binarization and calculating the soft prostate volume based on continuous weighting and integration. This aspect has an effect in that uncertainty in image-based volume estimation is reflected in risk assessment even for an identical PSA level by calculating both the confidence interval and the risk-weighted PSA density.

According to various embodiments of the present disclosure, voxels located at a boundary of the prostate region may comprise voxels in which probability distributions are mixed such that, within a local neighborhood of a voxel, values of the membership probability map supporting an interior of the prostate and values of the membership probability map supporting an exterior of the prostate coexist. The voxel-wise uncertainty map may comprise at least one of dispersion of voxel-wise membership probability values, predictive entropy, expected entropy, or a model uncertainty metric based on a difference therebetween, derived from results of multiple stochastic inferences of the probabilistic segmentation model or from uncertainty estimation of the model. The voxel-wise uncertainty map may be configured such that at least one of uncertainty caused by data noise and uncertainty caused by model parameters is represented as different uncertainty components. The continuous weighting function may be defined such that a voxel contribution monotonically increases as a value of the membership probability map increases and monotonically decreases as a value of the voxel-wise uncertainty map increases. The voxel contribution may include a normalization term configured to restrict the voxel contribution within a predefined range between a minimum contribution and a maximum contribution. The total accumulated uncertainty may comprise an aggregated value of uncertainty values of voxels contributing to calculation of the soft prostate volume. The confidence interval may comprise interval information including a lower bound volume value and an upper bound volume value determined based on a distribution of a plurality of soft prostate volume values derived from the multiple stochastic inferences of the probabilistic segmentation model. The risk-weighted prostate specific antigen density information may comprise a representative PSA density value calculated based on the serum prostate specific antigen level and the soft prostate volume, and at least one of a conservative PSA density value calculated using at least one of the lower bound volume value or the upper bound volume value of the confidence interval, or an uncertainty-based adjustment term combined with the representative PSA density value such that the representative PSA density value is adjusted more conservatively as the total accumulated uncertainty increases.

This aspect specifies boundary voxels of the prostate as mixed voxels in which an interior-supporting probability and an exterior-supporting probability coexist within a local neighborhood. This aspect specifies the uncertainty metric as at least one of dispersion, predictive entropy, expected entropy, or a model uncertainty metric based on a difference therebetween, and includes an uncertainty-component structure distinguishing a component caused by data noise and a component caused by model parameters.

This aspect has an effect of reducing interpretive ambiguity by collectively specifying boundary determination, uncertainty calculation, a monotonic condition of weighting, an aggregation scheme for the total accumulated uncertainty, a structure for deriving lower and upper bounds of the confidence interval, and representative and conservative values of the risk-weighted prostate specific antigen density information. This aspect structurally includes a mechanism in which risk weighting is automatically strengthened when uncertainty is high, thereby providing an effect in which a difference in image quality or model confidence is translated into a safety margin for clinical decision-making.

According to various embodiments of the present disclosure, the continuous weighting function may comprise a combination of a first monotonically increasing function with respect to values of the membership probability map and a second monotonically decreasing function with respect to values of the voxel-wise uncertainty map. The combination may include a first combination form having a saturation characteristic such that an influence of the second monotonically decreasing function is mitigated for voxels supported as belonging to an interior of the prostate, and a second combination form having a damping characteristic such that the influence of the second monotonically decreasing function is strengthened for voxels determined to be located at a boundary of the prostate. The continuous weighting function may be configured such that one of the first combination form or the second combination form is selected according to whether a voxel is located at the boundary.

This aspect specifies the continuous weighting function as a combined structure of a first monotonically increasing function for the membership probability map and a second monotonically decreasing function for the voxel-wise uncertainty map. This aspect includes a structure including a first combination form having a saturation characteristic and a second combination form having a damping characteristic, wherein one of the combination forms is selected according to whether a voxel is located at the boundary.

This aspect has an effect in that, even for an identical uncertainty value, the effect is reflected differently between voxels supported as belonging to an interior of the prostate and boundary voxels, thereby simultaneously ensuring stability of an interior region and conservativeness at the boundary. This aspect has an effect of suppressing excessive reflection of uncertainty at the boundary in voxel contribution to volume while reducing overestimation in sections where boundary ambiguity is high.

According to various embodiments of the present disclosure, the voxel-wise uncertainty map may comprise at least one of (a) a predictive entropy value for a predicted distribution of the membership probability map or (b) a mutual information value representing model uncertainty. The voxel-wise uncertainty map may be configured such that uncertainty caused by data noise and uncertainty caused by model parameters are separated and stored as different uncertainty components or represented as different channels. The continuous weighting function may be configured to receive at least one of the different uncertainty components according to different weighting application rules.

This aspect specifies the voxel-wise uncertainty map as uncertainty based on predictive entropy and mutual information. This aspect includes a structure in which uncertainty caused by data noise and uncertainty caused by model parameters are separated and stored as different uncertainty components or represented as different channels, and respective components are input to the continuous weighting function according to different rules.

This aspect has an effect in that interpretability and controllability are improved because types of causes of uncertainty are reflected in volume calculation in a separated state, as compared with an approach merely using the fact that uncertainty is high. This aspect has an effect in that robustness to changes in image acquisition environment or domain shift is improved because weighting reflection varies according to a relative proportion of noise-related uncertainty and model uncertainty.

According to various embodiments of the present disclosure, the confidence interval may be determined based on an empirical distribution of a plurality of soft prostate volume values generated by multiple stochastic inferences of the probabilistic segmentation model. The confidence interval may comprise interval information including a lower bound volume value and an upper bound volume value derived from the empirical distribution. The confidence interval may be defined to further include a representative value indicating a central tendency of the empirical distribution. The representative value may comprise at least one of a mean value, a median value, or a mode estimate of the plurality of soft prostate volume values.

This aspect includes a structure for calculating the confidence interval based on an empirical distribution of soft prostate volume values generated by multiple stochastic inferences. In this configuration, stochastic inference is an inference scheme that generates a plurality of results through different probabilistic paths for an identical input to form a distribution, and the empirical distribution is an observation-based distribution formed by those result values.

This aspect has an effect in that interval information including lower and upper bounds is provided together, unlike an approach providing only a single volume value, so that the magnitude of uncertainty is directly reflected in risk assessment. This aspect has an effect in that both provision of interval-based safety and convenience of single-indicator-based decision-making are ensured by further including a representative value.

According to various embodiments of the present disclosure, the risk-weighted prostate specific antigen density information may comprise a representative PSA density value calculated based on the serum prostate specific antigen level and a representative value of the soft prostate volume. The risk-weighted prostate specific antigen density information may further comprise a conservative PSA density value calculated using at least one of a lower bound volume value or an upper bound volume value of the confidence interval. The conservative PSA density value may be configured to reflect a prostate cancer risk more conservatively than the representative PSA density value.

This aspect includes a structure in which the risk-weighted prostate specific antigen density information includes both a representative PSA density value and a conservative PSA density value. This aspect is configured such that the conservative PSA density value is calculated using at least one of the lower bound volume value or the upper bound volume value of the confidence interval.

This aspect has an effect in that, even for an identical PSA level, if uncertainty in volume estimation is high, a conservative value is reflected in risk assessment, thereby reducing a risk of underestimation. This aspect has an effect of providing, in parallel, a reference value and a safety-margin value in clinical judgment through parallel provision of the representative value and the conservative value.

According to various embodiments of the present disclosure, the membership probability map may comprise probability values calibrated by a calibration function. The calibration function may be configured such that probability values of the membership probability map are statistically aligned with an observed occurrence frequency of prostate labels in validation data. The calibration function may comprise at least one of temperature scaling, isotonic regression, or Platt scaling. Probability values calibrated by the calibration function may preserve an order of probability values prior to calibration while reflecting a consistent label accuracy within identical probability intervals.

This aspect configures the membership probability map to include probability values calibrated by a calibration function. In this configuration, the calibration function is a function that corrects a probability scale such that predicted probabilities are statistically aligned with an actual label occurrence frequency, and temperature scaling, isotonic regression, and Platt scaling are implementation examples thereof.

This aspect has an effect in that meanings of probability values are aligned so as to better correspond to actual hit rates, thereby improving reliability of probability-based decision-making in subsequent uncertainty calculation or weighting calculation. This aspect has an effect in that interpretive consistency of identical probability intervals is improved while maintaining a relative comparison structure of classification performance through preservation of order.

According to various embodiments of the present disclosure, the soft prostate volume may comprise a consensus-based volume value corrected based on consistency of prostate boundary shapes derived from multiple directions including an axial plane, a sagittal plane, and a coronal plane. The consensus-based volume value may be configured such that, when prostate boundaries from the multiple directions do not coincide with each other, correction is preferentially concentrated on boundary regions to which higher uncertainty is assigned according to the voxel-wise uncertainty map. The consensus-based volume value may be configured as a corrected volume in which boundary conflicts are minimized such that volume deviation or boundary shape deviation among the multiple directions is reduced.

This aspect configures the soft prostate volume as a consensus-based volume based on consistency of boundary shapes derived from multiple directions including an axial plane, a sagittal plane, and a coronal plane. In this configuration, the consensus-based volume refers to a resultant volume obtained by reducing volume deviation or boundary deviation by adjusting mutually conflicting interpretations of boundaries when boundary inconsistency exists among multiple directions.

This aspect has an effect in that direction-dependent boundary fluctuation that may occur in segmentation based on a single direction is reduced, thereby improving reproducibility with respect to changes in image acquisition conditions. This aspect has an effect in that correction is preferentially allocated to boundary regions having higher uncertainty, thereby intensively mitigating volume error in regions where conflict is high.

According to various embodiments of the present disclosure, voxels located at a boundary of the prostate region may comprise voxels satisfying a criterion corresponding to a degree of mixture between membership probability values supporting an interior of the prostate and membership probability values supporting an exterior of the prostate within a local neighborhood of the voxel. The criterion for the boundary voxels may be determined by combining (i) a mixture degree according to distribution characteristics of membership probability values within the local neighborhood and (ii) at least one of a local variation or a local gradient of the membership probability map. The boundary voxels may be configured to be distinguishable from interior prostate voxels based on the combined determination.

This aspect configures a criterion for determining boundary voxels as a combination of a mixture degree based on local-neighborhood distribution and a local variation or a local gradient. This aspect structures boundary voxels to be distinguishable from interior prostate voxels as a result of the combined determination.

This aspect has an effect in that stability of boundary definition is improved by reflecting both a probabilistic structure of the boundary and a spatial variation structure thereof, as compared with simple threshold-based boundary determination. This aspect has an effect in that consistency of a volume calculation process is strengthened because boundary determination is configured in a form that is readily linked with the uncertainty map and the weighting function.

According to various embodiments of the present disclosure, the probabilistic segmentation model may comprise a three-dimensional convolutional neural network structure including an encoder and a decoder. The three-dimensional convolutional neural network structure may comprise a connection structure in which a plurality of multi-scale feature maps extracted by downsampling and a plurality of multi-scale feature maps restored by upsampling are combined by skip connections. The membership probability map may comprise values obtained by converting voxel-wise outputs of the decoder into probabilities by a sigmoid function or a softmax function. The probabilistic segmentation model may comprise a probabilistic component capable of generating a plurality of membership probability maps through stochastic inference such that variations in voxel-wise predicted distributions are reflected in the voxel-wise uncertainty map.

This aspect configures the probabilistic segmentation model as a three-dimensional convolutional neural network structure including an Encoder and a Decoder. In this configuration, a Skip Connection is a connection structure that directly combines downsampling feature maps and upsampling feature maps to compensate for loss of spatial information, and Sigmoid and Softmax are functions that convert voxel output values into probabilities.

This aspect has an effect in that boundary-shape restoration capability is improved through multi-scale feature combination, and a basis of the uncertainty map becomes clear because a plurality of membership probability maps are generated by the probabilistic component. This aspect has an effect in that explainability of uncertainty is improved because segmentation results and uncertainty calculation are coherently linked within an identical model structure.

According to various embodiments of the present disclosure, the membership probability map may comprise a probability field in which a probability of belonging to the prostate region is assigned as a continuous value corresponding to each voxel coordinate of the three-dimensional voxel data. The membership probability map may be stored in the memory in association with geometric metadata including spatial resolution information, image orientation information, and reference point information. The membership probability map may be managed together with the geometric metadata in a form in which a coordinate mapping relationship for the two-dimensional medical image data is maintained.

This aspect configures the membership probability map as a probability field corresponding to three-dimensional voxel coordinates and includes a structure in which the membership probability map is stored and managed in association with geometric metadata. In this configuration, the geometric metadata is auxiliary information including spatial resolution information, image orientation information, and reference point information, and enables the coordinate mapping relationship to be maintained.

This aspect has an effect in that registration between a coordinate system of an original image and a coordinate system of the probability map is maintained, thereby reducing coordinate distortion in overlay, boundary comparison, and follow-up comparison. This aspect has an effect in that, because coordinate mapping is maintained in a managed state, application of an identical standard is facilitated in subsequent visualization and long-term follow-up comparison.

According to various embodiments of the present disclosure, the voxel-wise uncertainty map may comprise, as at least one basic statistic, at least one of variance, standard deviation, or quantile-based dispersion of voxel-wise membership probability values calculated from a plurality of inference results of the probabilistic segmentation model. The voxel-wise uncertainty map may comprise values in which relatively large uncertainty values are mapped to voxels having large values of the basic statistic. The basic statistic included in the voxel-wise uncertainty map may be included in a form usable for deriving a lower bound volume value and an upper bound volume value constituting the confidence interval.

This aspect configures the voxel-wise uncertainty map to include at least one basic statistic among variance, standard deviation, and quantile-based dispersion. This aspect includes a structure in which the basic statistic is included in a form usable for deriving the lower bound volume value and the upper bound volume value of the confidence interval.

This aspect has an effect in that a numerical basis of uncertainty is explicitly presented as a statistic, thereby strengthening a link between the uncertainty map and the confidence interval. This aspect has an effect in that a basis for deriving the lower and upper bounds is maintained consistently with the empirical distribution, thereby improving stability and reproducibility of the confidence interval.

According to various embodiments of the present disclosure, the voxel-wise uncertainty map may comprise at least one of a local variation, a local gradient, or a local variance of the membership probability map among spatially adjacent voxels. The voxel-wise uncertainty map may comprise normalized values in which larger uncertainty values correspond to regions having a large local variation, the local gradient, or the local variance. The voxel-wise uncertainty map may include the local variation, the local gradient, or the local variance in a form usable for determining voxels located at a boundary of the prostate region.

This aspect configures the voxel-wise uncertainty map to include at least one of a local variation, a local gradient, or a local variance. This aspect includes a structure in which normalized values are included such that larger uncertainty values correspond to regions having large local characteristics, and such values are also included in a form usable for determining boundary voxels.

This aspect has an effect in that abrupt probability changes near the boundary are normalized and reflected as uncertainty, thereby reducing volume error due to excessive contribution of boundary voxels. This aspect has an effect in that consistency of a processing flow is strengthened because boundary determination and uncertainty calculation are linked based on the same local features.

According to various embodiments of the present disclosure, the soft prostate volume may comprise a value obtained by combining a physical volume of a unit voxel derived from spatial resolution metadata including pixel spacing information and slice thickness information of the three-dimensional voxel data, and a sum of voxel contributions reflecting the continuous weighting. The soft prostate volume may comprise a volume value in a form in which, even when the physical volume of the unit voxel differs by axis direction in medical image data having anisotropic resolution, a volume bias by axis direction is offset by a weighted sum of voxel contributions.

This aspect configures the soft prostate volume to include a value obtained by combining the physical volume of a unit voxel derived from spatial resolution metadata and a sum of voxel contributions reflecting the continuous weighting. This aspect includes a volume value in a form in which a volume bias by axis direction is offset by the weighted sum in anisotropic resolution.

This aspect has an effect in that volume bias occurring in anisotropic resolution is alleviated by a probability-based voxel-contribution summation structure, thereby improving comparability of volumes among data acquired under different imaging conditions. This aspect has an effect in that sensitivity to changes in spatial resolution is reduced by combining the physical volume and the sum of contributions, thereby improving stability of volume calculation.

According to various embodiments of the present disclosure, the confidence interval may comprise interval information including a representative value, a lower bound volume value, and an upper bound volume value of the soft prostate volume. The representative value may comprise at least one of a mean value, a median value, or a mode estimate of a plurality of soft prostate volume values generated by multiple stochastic inferences. The representative value may comprise a robust central value that maintains central tendency even when variation of some of the plurality of soft prostate volume values is excessive.

This aspect configures the confidence interval as interval information including a representative value, a lower bound volume value, and an upper bound volume value. This aspect includes a representative value comprising at least one of a mean value, a median value, or a mode estimate, and further comprising a robust central value that maintains central tendency even when some values exhibit excessive variation.

This aspect has an effect in that a representative value serving as a reference point in interval-based risk assessment is also provided, thereby improving clinical usability and consistency of decision-making. This aspect has an effect in that the representative value is maintained even when some outliers or unstable inference results are present, thereby improving practical stability of the confidence interval.

According to various embodiments of the present disclosure, the risk-weighted prostate specific antigen density information may comprise a representative PSA density value calculated based on the serum prostate specific antigen level and the representative value of the soft prostate volume. The risk-weighted prostate specific antigen density information may comprise a plurality of PSA density values respectively calculated corresponding to the lower bound volume value and the upper bound volume value of the confidence interval. PSA density range information may be constituted by the plurality of PSA density values.

This aspect configures the risk-weighted prostate specific antigen density information to include a representative PSA density value and a plurality of PSA density values respectively calculated corresponding to the lower bound volume value and the upper bound volume value. This aspect configures PSA density range information to be constituted by the plurality of PSA density values.

This aspect has an effect in that a risk of misjudgment in boundary cases that may occur in single-value-based determination is reduced and a safety margin for risk assessment is provided on a range basis. This aspect has an effect in that changes in the confidence interval of volume are directly transmitted to a PSA density range, thereby naturally transferring image-based uncertainty to a clinical indicator.

According to various embodiments of the present disclosure, the output risk-weighted prostate specific antigen density information may comprise at least one of overlay visualization information of a prostate region superimposable on the two-dimensional medical image data or heatmap visualization information expressing an uncertainty distribution in color. The visualization information may comprise pixel-wise or voxel-wise visualization values associated with the membership probability map or the voxel-wise uncertainty map in an identical coordinate system defined by geometric metadata. The heatmap visualization information may include, in a distinguishable manner, regions corresponding to relatively large uncertainty values in the voxel-wise uncertainty map.

This aspect configures the risk-weighted prostate specific antigen density information to include overlay visualization information or heatmap visualization information. In this configuration, the overlay is information displayed by superimposing visualization values related to the prostate region on original two-dimensional medical image data, and the heatmap is visualization information expressing an uncertainty distribution by color differences.

This aspect has an effect in that interpretation and verification of results become easier as compared with an approach providing only numerical indicators, thereby improving user reliability and suitability for a clinical workflow. This aspect has an effect in that regions having large uncertainty are displayed in a distinguishable manner, thereby improving allocation of attention and review efficiency of a reader.

According to various embodiments of the present disclosure, the output risk-weighted prostate specific antigen density information may comprise comparison metadata associated with at least one of a patient identifier, an examination identifier, image modality information, imaging parameter information, or PSA blood collection time-point information, such that examination results at a plurality of time points for an identical patient are traceable and comparable in a time-series manner. The comparison metadata may comprise registration information enabling the membership probability map, the voxel-wise uncertainty map, the soft prostate volume, and PSA density range information to be compared on an identical basis among examination results at the plurality of time points.

This aspect configures the comparison metadata to be associated with at least one of a patient identifier, an examination identifier, image modality information, imaging parameter information, or PSA blood collection time-point information. This aspect includes registration information enabling the membership probability map, the uncertainty map, the soft prostate volume, and the PSA density range information to be compared on an identical basis among examination results at multiple time points.

This aspect has an effect in that fluctuations in a comparison basis caused by differences in imaging conditions and time points are reduced in long-term follow-up, thereby improving reliability of time-series change interpretation. This aspect has an effect in that, because a registration structure of result data is included, data-management efficiency required for reproducibility verification, clinical research, and quality control is improved.

According to various embodiments of the present disclosure, the output risk-weighted prostate specific antigen density information may comprise information identifying, as a region of interest (ROI), a detailed region having relatively high uncertainty in the voxel-wise uncertainty map. The region-of-interest information may include a location of the detailed region and a slice index. The region-of-interest information may include at least one of an uncertainty value or an uncertainty component corresponding to the detailed region.

This aspect configures the risk-weighted prostate specific antigen density information to include information identifying a detailed region having high uncertainty as a region of interest (ROI). In this configuration, the region of interest (ROI) means region information for selecting, as a priority review target, a position centered on a relatively large uncertainty value or uncertainty component.

This aspect has an effect in that a spatial distribution of uncertainty is linked to reading priority, thereby improving both reading efficiency and safety within an identical period of time. This aspect has an effect in that an uncertainty value or component is provided together with the region of interest, thereby improving explainability as to why the corresponding region is a priority review target.

According to various embodiments of the present disclosure, voxels located at a boundary of the prostate region may comprise mixed boundary voxels determined based on a mixture degree of probability values within a local neighborhood of each voxel such that probability values of the membership probability map supporting an interior of the prostate and probability values supporting an exterior of the prostate coexist within the local neighborhood. The voxel-wise uncertainty map may comprise a first uncertainty component representing uncertainty caused by data noise and a second uncertainty component representing uncertainty caused by model parameters, wherein the first uncertainty component and the second uncertainty component are stored as different channels or different uncertainty terms. The continuous weighting function may be defined as a combined function including a first monotonically increasing function with respect to values of the membership probability map, a second monotonically decreasing function with respect to the first uncertainty component, a third monotonically decreasing function with respect to the second uncertainty component, and a shape-stability correction term derived from at least one of a local gradient consistency, a curvature variation between neighboring voxels, or a boundary alignment consistency between adjacent slices.

This aspect configures the continuous weighting function to include a combined structure reflecting both probability-based voxel membership and uncertainty-based reliability of the voxel prediction. In this configuration, the mixed boundary voxels correspond to voxels in which interior-supporting and exterior-supporting probabilities coexist within a local neighborhood, and the voxel-wise uncertainty map is structured to distinguish uncertainty caused by measurement noise from uncertainty caused by model parameters. The continuous weighting function is configured such that attenuation effects of uncertainty are mitigated for voxels supported as belonging to an interior region according to a saturation-type combination rule and strengthened for mixed boundary voxels according to a damping-type combination rule, thereby enabling voxel contributions used in calculation of the soft prostate volume to be normalized within a predefined contribution range.

This aspect has an effect in that reliability of boundary interpretation is improved because both probabilistic boundary mixture and uncertainty components are simultaneously reflected in the volume calculation process. This aspect has an effect in that stability of volume estimation is preserved in interior regions while uncertainty influence is selectively strengthened in boundary regions, thereby reducing volume overestimation or underestimation caused by ambiguous segmentation boundaries.

According to various embodiments of the present disclosure, the confidence interval of the soft prostate volume may be determined from an empirical distribution of a plurality of soft prostate volume values generated by multiple stochastic inferences of the probabilistic segmentation model. The confidence interval may comprise a representative volume value, a lower bound volume value, and an upper bound volume value. The risk-weighted prostate specific antigen density information may comprise a representative PSA density value calculated based on the serum prostate specific antigen level of the patient and the representative volume value, a conservative PSA density value calculated using at least one of the lower bound volume value or the upper bound volume value of the confidence interval, and an uncertainty-based adjustment term configured such that the representative PSA density value is shifted in a direction preventing underestimation of a prostate cancer risk as a total accumulated uncertainty increases.

This aspect configures the risk-weighted prostate specific antigen density information to include a representative PSA density value, a conservative PSA density value, and an uncertainty-based adjustment term derived from the confidence interval of the soft prostate volume. In this configuration, the confidence interval is derived from an empirical distribution obtained through multiple stochastic inferences of the probabilistic segmentation model, and the representative value corresponds to a central tendency of the empirical distribution, such as a mean value, a median value, or a mode estimate. The electronic device may further determine a clinical risk interpretation based on a positional relationship among the representative PSA density value, the conservative PSA density value, and a predefined clinical PSA density threshold.

This aspect has an effect in that uncertainty in image-based volume estimation is directly reflected in a clinical risk indicator rather than being merely presented as auxiliary information. This aspect has an effect in that a risk of underestimation of prostate cancer risk is reduced because conservative PSA density values and uncertainty-based adjustment terms are provided together with representative PSA density values, thereby enabling safer clinical decision-making when uncertainty is high.

According to various embodiments of the present disclosure, the soft prostate volume may comprise a consensus-based volume obtained by fusing a plurality of directional soft prostate volume candidate values respectively derived from segmentation results generated in an axial plane, a sagittal plane, and a coronal plane. The consensus-based volume may be determined based on directional reliability weights reflecting a boundary disagreement degree among the planes, a directional accumulated uncertainty derived from the voxel-wise uncertainty map, a local boundary continuity metric, and a reproducibility metric determined based on comparison with a prostate boundary obtained from a previous examination of the patient.

This aspect configures the soft prostate volume as a consensus-based volume derived from multi-directional segmentation results and reliability weighting. In this configuration, the reproducibility metric may be determined based on comparison metadata including at least one of a patient identifier, an examination identifier, image modality information, imaging parameter information, or a PSA blood-collection time-point identifier, such that segmentation results obtained at different examination time points can be compared within a common coordinate framework. The electronic device may adjust a width of the confidence interval according to directional reliability weights and the reproducibility metric and may further identify regions having relatively high uncertainty as regions of interest.

This aspect has an effect in that segmentation errors caused by direction-dependent boundary fluctuations are reduced because segmentation results obtained from multiple anatomical planes are fused based on reliability weights. This aspect has an effect in that reproducibility of prostate volume estimation across multiple examinations is improved and that regions having relatively high uncertainty are highlighted for review, thereby improving reliability and interpretability of diagnostic results.

According to various embodiments of The present disclosure, an electronic device for providing prostate substructure information may include an input device, an output device, a transceiver, a processor, and a memory, wherein the memory stores instructions for operations executed by the processor, and the operations may be configured to perform the method of operation of the electronic device according to the embodiment of FIG. 3A.

According to various embodiments of The present disclosure, a non-transitory computer-readable storage media storing instructions that, when executed by at least one processor, cause the at least one processor to perform operations may be provided, wherein the operations include the method of operation of the electronic device according to the embodiment of FIG. 3A.

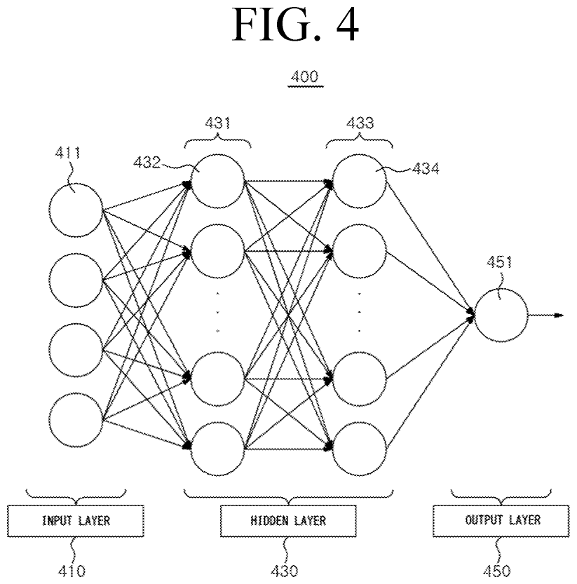

FIG. 4 illustrates a structure of a multi-layer perceptron (MLP) for implementing a deep learning algorithm according to various embodiments of the present disclosure.

According to various embodiments of the present disclosure, a prostate region analysis model may be configured as an MLP structure according to the embodiment of FIG. 4. The prostate region analysis model may be implemented using various architectures such as a convolutional neural network (CNN), DenseNet, U-Net, GoogLeNet, or a generative adversarial network. For example, when the prostate region analysis model is implemented as a CNN, the prostate region analysis model may perform a training process of adjusting connection weights of an artificial neural network using training data.

Deep learning is one of the technologies that has recently emerged in the field of machine learning, and is a neural network composed of a plurality of hidden layers and a plurality of hidden units included therein. When low-level features are input to a deep learning model, the low-level features are transformed, while passing through the plurality of hidden layers, into high-level features that can better describe a problem to be predicted. Since prior knowledge or intuition of an expert is not required in this process, subjective factors in feature extraction may be removed, and a model having a higher generalization capability may be developed. Furthermore, in deep learning, feature extraction and model construction are configured as one set, and thus a final model may be formed through a simpler process than in conventional machine learning theories.

A multi-layer perceptron (MLP) is a type of artificial neural network (ANN) having multiple nodes based on deep learning. Each node is a neuron similar to a biological connection pattern and uses a nonlinear activation function. This nonlinear property enables linearly inseparable data to be separated linearly.

Referring to FIG. 4, an artificial neural network 400 of an MLP model according to various embodiments of the present disclosure includes an input layer 410, a plurality of hidden layers 430, and an output layer 450.

Input data such as a plurality of factors related to medical images are input to nodes of the input layer 410. Here, the plurality of factors 411 related to the medical images correspond to low-level features of the deep learning model.

At nodes of the hidden layers 430, computations based on the input factors are performed. The hidden layers 430 are layers in which units defined as a plurality of nodes formed by combining the plurality of factors 411 related to the medical images are stored. As illustrated in FIG. 4, the hidden layers 430 may include a plurality of hidden layers.

For example, when the hidden layers 430 include a first hidden layer 431 and a second hidden layer 433, the first hidden layer 431 is a layer in which first units 432 defined as a plurality of nodes formed by combining the plurality of factors 411 related to the medical images, which are the lowest-level features, are stored, and each first unit 432 corresponds to a higher-level feature of the plurality of factors related to the medical images. The second hidden layer 433 is a layer in which second units 434 defined as a plurality of nodes formed by combining the first units of the first hidden layer 431 are stored, and each second unit 434 corresponds to a higher-level feature of the first units 432.

Nodes of the output layer 450 indicate calculated prediction results. The output layer 450 may include a plurality of prediction result units 451. Specifically, the plurality of prediction result units 451 may include two units, namely a True unit and a False unit. Specifically, the True unit is a prediction result unit indicating that a medical image is related to a specific feature, and the False unit is a prediction result unit indicating that the medical image is not related to the specific feature.

Weights may be assigned to respective connections between the second units 434 included in the second hidden layer 433, which is the last layer among the hidden layers 430, and the prediction result units 451. Based on these weights, whether the medical image is related to a specific feature may be predicted.

For example, when one of the second units 434 predicts that the medical image is related to a specific feature, the one of the second units 434 is connected to each of the True unit and the False unit, and a weight having a positive value may be assigned to the connection with the True unit, whereas a weight having a negative value may be assigned to the connection with the False unit. Conversely, when one of the second units 434 predicts that the medical image is not related to the specific feature, the one of the second units 434 is connected to each of the True unit and the False unit, and a weight having a negative value may be assigned to the connection with the True unit, whereas a weight having a positive value may be assigned to the connection with the False unit.

A plurality of connection lines may be formed between the plurality of second units 434 and the True unit. When a total sum of the plurality of connection lines has a positive value, the plurality of factors 411 related to the medical image in the input layer 410 may be predicted as factors indicating that the medical image is related to the specific feature. According to an embodiment, whether the medical image is related to the specific feature may also be predicted by comparing the total sum of the plurality of connection lines with a preset value.

The artificial neural network 400 of the MLP model learns by adjusting training parameters. According to an embodiment, the training parameters include at least one of weights and biases. The training parameters are repeatedly adjusted through an optimization algorithm called gradient descent. Each time a prediction result is calculated from a given data sample by forward propagation, performance of the network is evaluated through a loss function measuring prediction error. Each training parameter of the artificial neural network 400 is adjusted by slightly increasing or decreasing the parameter in a direction that minimizes a value of the loss function, and this process is referred to as back-propagation.

The present disclosure provides a software program for analyzing CT and MRI images, measuring a volume of a prostate, and visually displaying a prostate region, with an objective of assisting a user in performing diagnosis of prostate disease more efficiently.

The present disclosure supports DICOM and NIFTI standards, which are mainly used in medical devices and Picture Archiving and Communication System (PACS) software, and intuitively provides a prostate volume and a prostate region to a user by visualizing multi-slice medical images in two dimensions and three dimensions.

The present disclosure further calculates PSA density based on a prostate volume in cubic centimeters and a serum PSA level in ng/mL, and determines that a likelihood of prostate cancer is high when the PSA density is 0.15 ng/mL/cc or more.

The present disclosure assists a user in performing a prostate cancer diagnosis procedure more conveniently and accurately by automating a prostate volume measurement process and a visualization process.

This software is responsible for measuring an overall prostate volume. The overall prostate volume is used for PSA density calculation, and measurement of sub-regions is used to evaluate whether benign prostatic hyperplasia is present. Detection of a prostate cancer region supports the user in easily performing diagnosis by intuitively visualizing a prostate portion in a plurality of CT and MRI images.

Prostate cancer is a disease in which the number of patients has steadily increased due to aging and the spread of Western dietary habits. According to the Health Insurance Review and Assessment Service of Korea, the number of prostate cancer patients increased by about 41.56%, from 85,943 in 2018 to 121,665 in 2022, and medical expenses increased by 52.8% during the same period. This further highlights a need for early diagnosis and efficient management of prostate cancer.

In diagnosis of prostate cancer, prostate ultrasound examination has become an essential tool, but is currently limited mainly to measurement of prostate volume. In addition, an examination cost of approximately KRW 150,000 at a tertiary general hospital may impose an economic burden.

The global MRI market was valued at approximately USD 5.32 billion in 2022, and is projected to grow at a compound annual growth rate of 5.9% from 2023 to 2030. In particular, an AI diagnostic market using AI algorithms is rapidly growing and is expected to reach USD 11.9 billion by 2030.

Applicability of AI-based medical devices is gradually expanding, and revision of national health insurance listing guidelines and recognition of reimbursement for interpretation further increase stability and potential value of such technologies.

In a current medical environment in which CT and MRI imaging is commonplace, prostate ultrasound examination may cause waste of health insurance finances due to duplicate examinations.

As of 2023, approximately 17,648 CT devices and 1,999 MRI devices were installed in Korea, and expansion in dissemination of such medical equipment and application of AI technology are leading growth of a medical market and improvement in diagnostic efficiency.

Such a technical background suggests that application of AI technology based on CT and MRI image data may be an important alternative in diagnosis of prostate cancer.

In this situation, the present disclosure provides an automated diagnostic support system capable of improving efficiency of prostate cancer diagnosis, addressing a duplicate examination issue, and precisely detecting a cancer-suspected region using CT and MRI data.

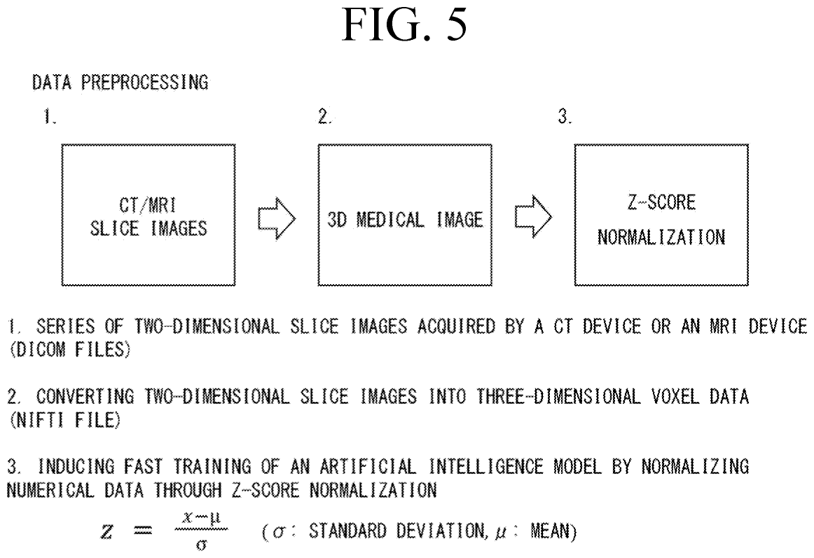

FIG. 5 illustrates an example of a preprocessing process of image data before the image data is input to an artificial intelligence model according to various embodiments of the present disclosure.

FIG. 5 illustrates a preprocessing process of image data according to various embodiments of the present disclosure.

In a first stage, a plurality of two-dimensional (2D) slice images including a prostate region of a patient are acquired using a CT device or an MRI device. MRI and CT images are captured as multiple slices, and typically each slice is converted into and stored as a single DICOM file. These images are generally stored in a DICOM (Digital Imaging and Communications in Medicine) format and form an image data series as a collection of slice images.

In a second stage, the two-dimensional slice image data are converted into three-dimensional medical image data. The segmented DICOM files are combined into a single file to construct three-dimensional voxel data and are then converted into a NIFTI file. In this process, a concept of voxel data is used. A voxel represents an extension of a pixel unit into three dimensions and constitutes volumetric data. The converted data are stored in a NIFTI (NIfTI-1) file format, which can be efficiently utilized in various medical image analysis software applications.

In a third stage, numerical data are refined through Z-score normalization. Before being input into an artificial intelligence model, the converted NIFTI file undergoes Z-score normalization to normalize the numerical data into a normal distribution. In medical imaging data, pixel values may vary depending on imaging equipment or acquisition conditions, which may make accurate training of an artificial intelligence model difficult. Z-score normalization resolves such imbalance in the data and performs normalization so that all pixel values fall within the same distribution. The normalized data facilitate faster and more stable training of the artificial intelligence model.

The Z-score normalization process may be performed as follows. Z-score normalization is performed by subtracting a mean value μof the data from each pixel value x and dividing the result by a standard deviation σ. Through this process, the data follow a normal distribution having a mean of 0 and a standard deviation of 1. This may be expressed by the following equation:

Z=(x−μ)/Σ.

Through this normalization process, the data are adjusted to a consistent range across different images, enabling the artificial intelligence model to learn the data more efficiently.

Through Z-score normalization, the numerical data are refined into a form close to a normal distribution while reducing noise. In particular, imbalance in pixel values in medical images is resolved, thereby improving data quality and significantly enhancing both the training speed and accuracy of the artificial intelligence model. As a result, base data required for measurement of a total prostate volume and detection of a prostate region are optimized.

The normalized data that have undergone the preprocessing process are subsequently input to the artificial intelligence model. Based on these data, the model measures a total volume of the prostate and detects the prostate region. Such preprocessing serves as a critical step that enhances reliability of prostate cancer diagnosis by ensuring accuracy and consistency of the data.

Since three-dimensional images require a large amount of computation and significantly increase inference time of an artificial intelligence model, a location of the prostate is first estimated from the entire MRI or CT image set, and only a corresponding region is cropped and used as an input to the artificial intelligence model.

For example, when an upper-body CT image having a resolution of 512×512 is composed of 200 slices, slices including the prostate typically correspond to about 20 to 30 slices. In such a case, slices are cropped around the prostate region by adding margin padding of approximately −10 to +10 slices before and after the prostate region, and the cropped images are converted into a three-dimensional NIFTI file.

A method of locating the prostate region includes slicing the entire three-dimensional image dataset in frontal and lateral directions and applying a two-dimensional object detection model to precisely identify a location of the prostate. Through this approach, only necessary portions of the image are efficiently extracted, thereby reducing computation time and optimizing performance of the artificial intelligence model.

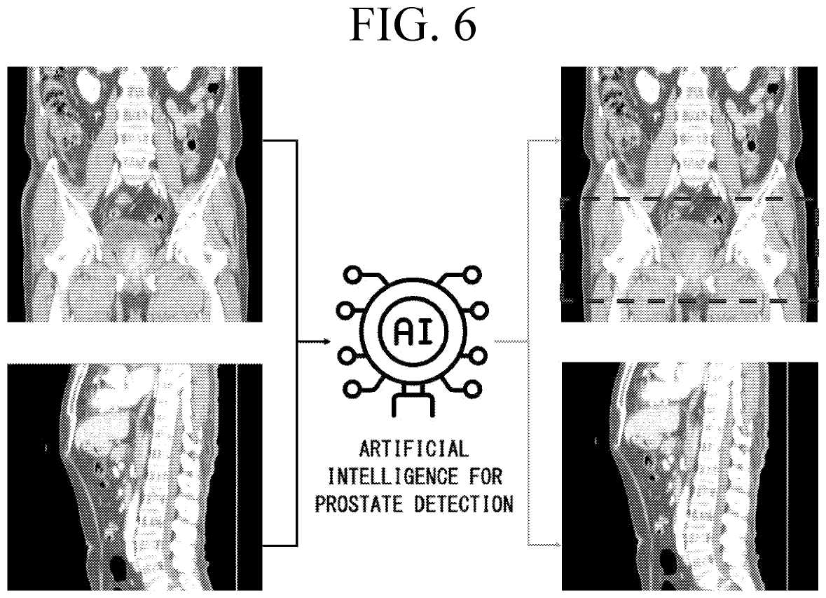

FIG. 6 illustrates an example of an artificial intelligence model for detecting a prostate region according to various embodiments of the present disclosure.

FIG. 6 illustrates an example of a process in which an artificial intelligence model for detecting a prostate region analyzes CT and MRI image data to detect the prostate region according to various embodiments of the present disclosure. The artificial intelligence model precisely detects the prostate region from the input medical image data, visually distinguishes the prostate structure, and displays the result to a user.

The artificial intelligence model of the present disclosure receives, as input, a plurality of slice images acquired from a CT device or an MRI device. The anterior-posterior and lateral CT images shown on the left side correspond to original input data. The data are composed of two-dimensional slice images, and the prostate region is detected based on these images.

To detect a location and shape of the prostate in the input image data, the artificial intelligence model applies a prostate detection algorithm. The artificial intelligence (AI) model illustrated in the center employs a deep learning-based model (e.g., a convolutional neural network (CNN)) trained to learn features of the prostate region, thereby automatically performing segmentation of the prostate and identifying cancer-suspected regions.

As illustrated in the upper right image of FIG. 6, after detecting the prostate region, the artificial intelligence model identifies a cancer-suspected region. In the figure, a portion indicated in green represents a cancer-suspected region detected by the AI model, and the region is distinguished from surrounding normal tissue using techniques such as texture analysis or pixel value comparison.

In addition, a dotted box shown in FIG. 6 emphasizes a region of interest (ROI) in which the detected prostate region is located.

The lower right image of FIG. 6 presents a side-view example in which the prostate region and the cancer-suspected region are visualized from a different angle. The artificial intelligence model converts the detected results into two-dimensional (2D) and three-dimensional (3D) visualization data and provides the visualization data to the user, thereby assisting a diagnostician in intuitively identifying cancer-suspected regions.

The artificial intelligence model of the present disclosure can automatically analyze a large number of CT or MRI slice images to detect the prostate region quickly and accurately. As a result, the diagnosis time required by medical personnel may be reduced and diagnostic accuracy may be improved. At the same time, redundant examinations may be reduced, thereby decreasing medical costs for patients and medical institutions. In particular, the visualized results may serve as an important auxiliary tool in a diagnostic process for prostate-related diseases.

### Artificial Intelligence Segmentation Processing

The artificial intelligence model of the present disclosure includes a model for measuring a prostate volume.

Conventional artificial intelligence models typically apply a two-dimensional (2D) segmentation method that processes sliced medical images individually and masks a prostate object region on a slice-by-slice basis.

However, although the prostate region exhibits a relatively consistent pattern when represented in three dimensions, the prostate region appears irregular and discontinuous in individual two-dimensional slice images. Due to this characteristic, conventional artificial intelligence models may encounter limitations in accurately learning the prostate structure.

According to the present disclosure, MRI and CT images are converted into three-dimensional form, and a three-dimensional convolutional neural network (3D CNN) is applied to accurately mask the prostate object region. Through this approach, both a total prostate volume and a prostate region can be detected with higher precision.

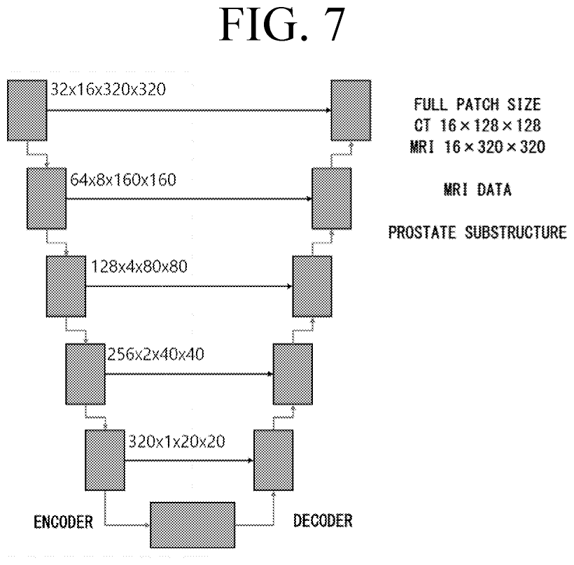

FIG. 7 illustrates an example of a 3D U-Net structure (MRI image input example) according to various embodiments of the present disclosure.

FIG. 7 illustrates an example of MRI image input using a 3D U-Net structure according to various embodiments of the present disclosure. The artificial intelligence model used in the present disclosure is configured as a model for measuring a prostate volume.

Conventional two-dimensional (2D) segmentation models process medical images slice by slice, and therefore have limitations in learning efficiency due to the irregular appearance of the prostate in individual slices. In contrast, when the prostate is converted into a three-dimensional form, it exhibits a relatively consistent structural pattern, enabling the artificial intelligence model to perform more precise learning.

Accordingly, segmentation processing using a 3D U-Net artificial intelligence architecture may be applied. In the present disclosure, MRI and CT image data are converted into a three-dimensional form, and a three-dimensional convolutional neural network (3D CNN) is used to mask a prostate object region. Through this approach, the location and shape of the prostate structure can be detected more accurately.

The application sequence of the artificial intelligence model may be described as follows.

In a first stage, preprocessing of CT and MRI images is performed, and a full image dataset having a size of 512×512×(number of slices) is prepared for input into the 3D U-Net model.

In a second stage, the image is divided into patches and provided as input to the model. In one example, a patch size of MRI images is 16×320×320, and a patch size of CT images is 16×128×128.

In a third stage, with respect to an encoder and a decoder of the 3D U-Net, the encoder extracts feature points from the input images, and the decoder converts the extracted features into images masked as prostate object regions. In this process, the encoder of the U-Net structure progressively reduces image resolution while extracting features, and the decoder restores the image to an original resolution using the extracted features.

In a fourth stage, a mask image is generated. Specifically, a mask image having the same resolution as the input image, namely 512×512×(number of slices), is generated. The mask is a binary image composed of values of 0 and 1, and pixels having a value of “1” represent a prostate object.

In a fifth stage, a prostate volume is calculated. Pixel information of the generated mask image is converted into a volumetric value expressed in cubic centimeters (cc), thereby calculating the prostate volume. In this process, pixel size information included in DICOM and NIFTI files is used to derive an actual physical volume.

The finally derived prostate volume is output by the program in a visualized form. Through such visualization, a user can intuitively identify the condition of the prostate and can perform diagnosis of prostate-related diseases in a more precise and efficient manner.

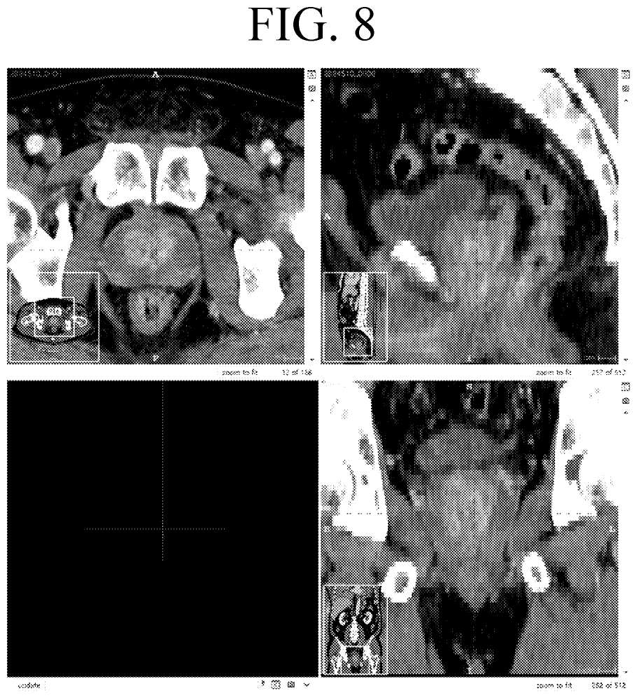

FIG. 8 illustrates an example of an output screen of an electronic device for providing prostate detail information based on an artificial intelligence model according to various embodiments of the present disclosure.

FIG. 8 illustrates an example of an output screen of an electronic device for providing prostate detailed information based on an artificial intelligence model according to various embodiments of the present disclosure. The present disclosure includes a function of automatically detecting a prostate region using CT or MRI medical images and quantitatively analyzing the detected region to calculate a prostate volume and a prostate specific antigen density (PSA density). The output screen is designed to simultaneously visualize medical images in multiple planes including axial, sagittal, and coronal planes so that a user can intuitively confirm a shape of the prostate and analysis results. Through such visualization, diagnosis and progression of prostate cancer can be evaluated more precisely.

The output screen is composed of four windows, among which three windows provide meaningful analysis data. A first window located at an upper left displays a prostate image based on an axial plane, in which the prostate is highlighted in a specific color, for example red. In this window, an anatomical position of the prostate can be confirmed, and it can be reviewed whether an automatically detected prostate region has been correctly identified. In addition, the axial plane enables an overall shape of the prostate and a relationship with surrounding tissues to be identified at a glance, which is useful for analyzing characteristics of a region suspected of prostate cancer.

A second window located at an upper right displays a medical image based on a coronal plane so that a vertical shape of the prostate can be confirmed. In this window, the prostate is highlighted in a specific color such as red, allowing a relative position of the prostate within a body of a patient to be clearly identified. In particular, the coronal plane enables analysis of a superior inferior length and an internal structure of the prostate and facilitates identification of a specific lesion when such lesion is present within the prostate.

A third window located at a lower right displays a medical image based on a sagittal plane and is useful for precisely analyzing an anterior posterior length and an internal structure of the prostate. In this window, the prostate is highlighted in a specific color such as red, enabling morphological characteristics of the prostate and relationships with surrounding organs, for example a bladder and a rectum, to be easily evaluated. The sagittal plane may be particularly useful for monitoring a change in prostate size or determining whether benign prostatic hyperplasia (BPH) is present.

In an embodiment of the present disclosure, a prostate volume is precisely measured based on three dimensional voxel data, and PSA density is calculated by combining the measured volume with a serum PSA level of a patient. Through this approach, medical personnel can more intuitively evaluate a spatial structure and size of the prostate and can utilize the information to comprehensively analyze a prostate condition of the patient.

The system supports medical personnel in performing faster and more accurate diagnosis by using an artificial intelligence based automatic analysis function. In particular, the output screen configuration of the present disclosure provides various structural information of the prostate through multi plane analysis and enables intuitive confirmation of a prostate volume through three dimensional reconstruction. As a result, more efficient and reliable results can be provided compared with conventional manual analysis methods. Through this configuration, early diagnosis of prostate cancer and establishment of treatment plans can be facilitated, thereby maximizing practical usability in clinical environments.

### 1. Background Technology of the Present Disclosure

The present disclosure automatically performs precise segmentation and volume measurement of the prostate using high resolution CT and MRI images. In this process, a deep learning algorithm, particularly a convolutional neural network (CNN), is used to accurately identify a boundary of the prostate and convert an overall shape and structure of the prostate into three dimensional voxel data. Prostate volume and PSA density are important indicators in diagnosing prostate cancer. This automated approach reproduces detailed structures of the prostate with high accuracy and provides data that are significantly faster and more accurate than manual measurements performed by medical professionals, thereby improving clinical diagnostic efficiency.

### 2. Objective of the Present Disclosure

An objective of the present disclosure is to accurately measure a prostate volume from medical images and calculate PSA density based on the measured volume in order to improve early diagnosis of prostate cancer and monitoring of disease progression.

### 3. Implementation Method of the Present Disclosure

### (1) Data Input and Processing

CT or MRI data in DICOM or NIFTI format are input and converted into three dimensional voxel data.

### (2) Image Normalization

Image data are standardized using Z score normalization. (3) Prostate region detection

A deep learning model based on a convolutional neural network automatically detects boundaries of the prostate.

### (4) Volume Measurement

A precise prostate volume is measured based on voxels of the detected prostate region.

### (5) PSA density calculation

PSA density is calculated by combining the measured prostate volume with a serum PSA level of the patient.

### (6) Result Visualization

Two dimensional and three dimensional visualization techniques are used to display prostate volume and PSA density results.

### (7) Report Generation

A comprehensive analysis report is automatically generated to support diagnosis by medical professionals.

### 4. Implementation Results of the Present Disclosure

### Results of Prostate Segmentation and Volume Measurement Based on CT and MRI

The present disclosure automatically performs segmentation and volume measurement of the prostate using CT and MRI images. By applying deep learning technology, particularly convolutional neural networks, boundaries of the prostate are precisely identified and a shape of the prostate is accurately reconstructed using three dimensional voxel data. Images obtained through this process visualize structural details of the prostate in high resolution and provide higher precision and repeatability compared with manual measurement methods.

### Main Features of the Present Disclosure

The present disclosure relates to a method and apparatus for automatically measuring a total volume of a prostate from CT or MRI medical images using artificial intelligence technology and calculating PSA density using the measured volume. By automatically detecting a prostate region in medical images and calculating a precise volume, the present disclosure enables more accurate calculation of PSA density. Conventional manual prostate volume measurement methods have large inter examiner variations and involve complicated calculation processes. In contrast, the present disclosure enables more consistent and accurate measurement through artificial intelligence based automation.

The present disclosure includes the following major processes.

### 1. Medical image data input and preprocessing

(1) CT or MRI image data are received in DICOM or NIFTI format and converted into three dimensional voxel data.

(2) Z score normalization is performed to standardize the data.

### 2. Prostate region detection and volume measurement

(1) A deep learning based model automatically detects boundaries of the prostate.

(2) Multi plane analysis including axial, sagittal, and coronal planes is used to analyze a prostate shape more precisely.

(3) Three dimensional volume rendering is applied to visually represent an entire shape of the prostate.

(4) Precise voxel based volume measurement is performed based on the detected prostate region.

### 3. PSA Density Calculation and Comparative Analysis

(1) A serum PSA level of the patient is input and combined with the prostate volume to automatically calculate PSA density.

(2) The calculated value is compared with normal ranges and clinical criteria to evaluate a likelihood of prostate cancer.

### 4. Result Visualization and Report Generation

(1) Two dimensional and three dimensional visualization are used to intuitively present a prostate shape and volume measurement results.

(2) PSA density results are presented together with reference ranges.

(3) A comprehensive analysis report supporting clinical diagnosis is automatically generated.

In a case where the embodiments of The present disclosure are implemented using hardware, Application Specific Integrated Circuits (ASICs), Digital Signal Processors (DSPs), Digital Signal Processing Devices (DSPDs), Programmable Logic Devices (PLDs), Field Programmable Gate Arrays (FPGAs), or the like, configured to perform The present disclosure, may be provided in the processor of The present disclosure.

Meanwhile, the above-described method can be written as a program executable on a computer, and can be implemented in a general-purpose digital computer that operates the program using a computer-readable medium. In addition, the structure of data used in the above-described method may be recorded on a computer-readable storage medium through various means. Program storage devices usable to describe a storage device including executable computer code to perform the various methods of The present disclosure should not be understood as including transitory subjects such as carrier waves or signals. The computer-readable storage medium includes storage media such as magnetic storage media (e.g., ROM, floppy disk, hard disk, etc.) and optical reading media (e.g., CD-ROM, DVD, etc.).

The embodiments described above are combinations of elements and features of The present disclosure in a predetermined form. Each element or feature should be considered optional unless explicitly stated otherwise. Each element or feature may be implemented in a form not combined with other elements or features. Also, it is possible to configure an embodiment of The present disclosure by combining some elements and/or features. The order of operations described in the embodiments of The present disclosure may be changed. Some configurations or features of one embodiment may be included in other embodiments, or may be replaced with corresponding configurations or features of other embodiments. It is apparent that claims that do not have an explicit citation relationship in the claims may be combined to configure an embodiment or may be included as a new claim by amendment after filing.

It will be apparent to those skilled in the art that The present disclosure may be embodied in other specific forms without departing from the technical spirit and essential characteristics of The present disclosure. Therefore, the above embodiments should be considered in all respects as illustrative and not restrictive. The scope of The present disclosure should be determined by reasonable interpretation of the appended claims and all possible changes within the equivalent scope of The present disclosure.

What is claimed is:

1. A method performed by an electronic device, the electronic device comprising an input device, an output device, a processor, and a memory, the method comprising:
acquiring, by the input device, two-dimensional medical image data related to a prostate of a patient from a magnetic resonance imaging (MRI) device or a computed tomography (CT) device;
generating, by the processor, three-dimensional voxel data of the prostate based on the two-dimensional medical image data;
applying, by the processor, a probabilistic segmentation model to the three-dimensional voxel data to generate (i) a membership probability map representing, for each voxel, a probability that the voxel belongs to a prostate region as a continuous value between 0 and 1, and (ii) a voxel-wise uncertainty map representing a degree of prediction uncertainty of the membership probability map;
calculating, by the processor, a soft prostate volume by integrating continuous values of the membership probability map using a continuous weighting function defined such that (i) the weighting function is proportional to values of the membership probability map and (ii) the weighting function is inversely proportional to values of the voxel-wise uncertainty map, for voxels located at a boundary of the prostate region without performing threshold-based binarization;
determining, by the processor, a confidence interval of the calculated soft prostate volume based on a total amount of uncertainty accumulated during calculation of the soft prostate volume;
generating, by the processor, risk-weighted prostate specific antigen density (Risk-Weighted PSAD) information serving as a criterion for assessing a prostate cancer risk, based on a serum prostate specific antigen level of the patient and the soft prostate volume reflecting the confidence interval; and
outputting, by the output device, the risk-weighted prostate specific antigen density information.

2. The method of claim 1,
wherein voxels located at a boundary of the prostate region comprise voxels in which probability distributions are mixed such that, within a local neighborhood of a voxel, values of the membership probability map supporting an interior of the prostate and values of the membership probability map supporting an exterior of the prostate coexist;
wherein the voxel-wise uncertainty map comprises at least one of dispersion of voxel-wise membership probability values, predictive entropy, expected entropy, or a model uncertainty metric based on a difference therebetween, the voxel-wise uncertainty map is derived from results of multiple stochastic inferences of the probabilistic segmentation model or from uncertainty estimation of the model, and the voxel-wise uncertainty map is configured such that at least one of uncertainty caused by data noise and uncertainty caused by model parameters is represented as different uncertainty components;
wherein the continuous weighting function is defined such that a voxel contribution monotonically increases as a value of the membership probability map increases and monotonically decreases as a value of the voxel-wise uncertainty map increases, and the voxel contribution includes a normalization term configured to restrict the voxel contribution within a predefined range between a minimum contribution and a maximum contribution;
wherein the total accumulated uncertainty comprises an aggregated value of uncertainty values of voxels contributing to calculation of the soft prostate volume;
wherein the confidence interval comprises interval information including a lower bound volume value and an upper bound volume value determined based on a distribution of a plurality of soft prostate volume values derived from the multiple stochastic inferences of the probabilistic segmentation model; and
wherein the risk-weighted prostate specific antigen density (Risk-Weighted PSAD) information comprises:
(i) a representative PSA density value calculated based on the serum prostate specific antigen level and the soft prostate volume, and
(ii) at least one of a conservative PSA density value calculated using at least one of the lower bound volume value or the upper bound volume value of the confidence interval, or an uncertainty-based adjustment term combined with the representative PSA density value such that the representative PSA density value is adjusted more conservatively as the total accumulated uncertainty increases.

3. The method of claim 1,
wherein the continuous weighting function comprises a combination of a first monotonically increasing function with respect to values of the membership probability map and a second monotonically decreasing function with respect to values of the voxel-wise uncertainty map,
wherein the combination includes a first combination form having a saturation characteristic such that an influence of the second monotonically decreasing function is mitigated for voxels supported as belonging to an interior of the prostate, and a second combination form having a damping characteristic such that the influence of the second monotonically decreasing function is strengthened for voxels determined to be located at a boundary of the prostate, and
wherein the continuous weighting function is configured such that one of the first combination form or the second combination form is selected according to whether a voxel is located at the boundary.

4. The method of claim 1,
wherein the voxel-wise uncertainty map comprises at least one of (a) a predictive entropy value for a predicted distribution of the membership probability map or (b) a mutual information value representing model uncertainty,
wherein the voxel-wise uncertainty map is configured such that uncertainty caused by data noise and uncertainty caused by model parameters are separated and stored as different uncertainty components or represented as different channels, and
wherein the continuous weighting function is configured to receive at least one of the different uncertainty components according to different weighting application rules.

5. The method of claim 1,
wherein the confidence interval is determined based on an empirical distribution of a plurality of soft prostate volume values generated by multiple stochastic inferences of the probabilistic segmentation model,
wherein the confidence interval comprises interval information including a lower bound volume value and an upper bound volume value derived from the empirical distribution, and
wherein the confidence interval is defined to further include a representative value indicating a central tendency of the empirical distribution, the representative value comprising at least one of a mean value, a median value, or a mode estimate of the plurality of soft prostate volume values.

6. The method of claim 1,
wherein the risk-weighted prostate specific antigen density information comprises a representative PSA density value calculated based on the serum prostate specific antigen level and a representative value of the soft prostate volume,
wherein the risk-weighted prostate specific antigen density information further comprises a conservative PSA density value calculated using at least one of a lower bound volume value or an upper bound volume value of the confidence interval, and
wherein the conservative PSA density value is configured to reflect a prostate cancer risk more conservatively than the representative PSA density value.

7. The method of claim 1,
wherein the membership probability map comprises probability values calibrated by a calibration function,
wherein the calibration function is configured such that probability values of the membership probability map are statistically aligned with an observed occurrence frequency of prostate labels in validation data,
wherein the calibration function comprises at least one of temperature scaling, isotonic regression, or Platt scaling, and
wherein probability values calibrated by the calibration function preserve an order of probability values prior to calibration while reflecting a consistent label accuracy within identical probability intervals.

8. The method of claim 1,
wherein the soft prostate volume comprises a consensus-based volume value corrected based on consistency of prostate boundary shapes derived from multiple directions including an axial plane, a sagittal plane, and a coronal plane,
wherein, when prostate boundaries from the multiple directions do not coincide with each other, the consensus-based volume value is configured such that correction is preferentially concentrated on boundary regions to which higher uncertainty is assigned according to the voxel-wise uncertainty map, and
wherein the consensus-based volume value is configured as a corrected volume in which boundary conflicts are minimized such that volume deviation or boundary shape deviation among the multiple directions is reduced.

9. The method of claim 1,
wherein voxels located at a boundary of the prostate region comprise voxels satisfying a criterion corresponding to a degree of mixture between membership probability values supporting an interior of the prostate and membership probability values supporting an exterior of the prostate within a local neighborhood of the voxel,
wherein the criterion for the boundary voxels is determined by combining (i) a mixture degree according to distribution characteristics of membership probability values within the local neighborhood and (ii) at least one of a local variation or a local gradient of the membership probability map, and
wherein the boundary voxels are configured to be distinguishable from interior prostate voxels based on the combined determination.

10. An electronic device comprising:
an input device; an output device; a processor; and a memory storing instructions executable by the processor,
wherein the instructions cause the processor to perform operations comprising:
acquiring, by the input device, two-dimensional medical image data related to a prostate of a patient from a magnetic resonance imaging (MRI) device or a computed tomography (CT) device;
generating, by the processor, three-dimensional voxel data of the prostate based on the two-dimensional medical image data;
applying, by the processor, a probabilistic segmentation model to the three-dimensional voxel data to generate (i) a membership probability map representing, for each voxel, a probability that the voxel belongs to a prostate region as a continuous value between 0 and 1, and (ii) a voxel-wise uncertainty map representing a degree of prediction uncertainty of the membership probability map;
calculating, by the processor, a soft prostate volume by integrating continuous values of the membership probability map using a continuous weighting function defined such that (i) the weighting function is proportional to values of the membership probability map and (ii) the weighting function is inversely proportional to values of the voxel-wise uncertainty map, for voxels located at a boundary of the prostate region without performing threshold-based binarization;
determining, by the processor, a confidence interval of the calculated soft prostate volume based on a total amount of uncertainty accumulated during calculation of the soft prostate volume;
generating, by the processor, risk-weighted prostate specific antigen density (Risk-Weighted PSAD) information serving as a criterion for assessing a prostate cancer risk, based on a serum prostate specific antigen level of the patient and the soft prostate volume reflecting the confidence interval; and
outputting, by the output device, the risk-weighted prostate specific antigen density information.
